(19) United States
(12) Patent Application Publication
(10) Pub. No.: US 2013/0224106 A1
(43) Pub. Date: Aug. 29, 2013

(54) HYDROGEN GENERATION

(71) Applicant: King Abdullah University of Science and Technology, (US)

(72) Inventors: Jean-Marie BASSET, Thuwal (SA); Vivek POLSHETTIWAR, Thuwal (SA); Mohamed BOUHRARA, Thuwal (SA); Youssef SAIH, Thuwal (SA)

(73) Assignee: KING ABDULLAH UNIVERSITY OF SCIENCE AND TECHNOLOGY, Thuwal (SA)

(21) Appl. No.: 13/746,936

(22) Filed: Jan. 22, 2013

Related U.S. Application Data
(60) Provisional application No. 61/589,689, filed on Jan. 23, 2012.

(51) Int. Cl. C01B 3/26; C07C 2/76

(52) U.S. Cl. CPC C01B 3/26; C07C 2/76
USPC 423/651; 585/700

(57) ABSTRACT

A process for the decomposition of methane can be controlled to form ethane or hydrogen with a solid carbon product.

### CLAIM FOR PRIORITY

This application claims priority to U.S. Patent Application No. 61/589,689, filed Jan. 23, 2012, which is hereby incorporated by reference in its entirety.

### TECHNICAL FIELD

This invention relates to controlling production of hydrogen gas or ethane from methane.

### BACKGROUND

There is a fantastic energy issue facing the world, due to the increase of population resulting in the increase of energy demand and conflicting with planet sustainability. The perspective scenario regarding ecology driven policy can't be fulfilled by fossil fuel after 2130 with the existing energy strategies.

It is known that proven reserves of natural gas in the world are increasingly surpassing the proven reserves of petroleum. Besides natural gas being a gas, it is more easily extracted from the ground from liquid or viscous liquid. Nobody so far has been thinking that gas could be a clean source of hydrogen with low carbon footprint. Presently hydrogen is produced by steam reforming of methane but it produces a huge amount of carbon dioxide. As a matter of fact, in the steam reforming of methane, 1 Ton of hydrogen emits 9 Tons of CO₂. Beside this very high CO₂ emission, further purification steps are required to separate hydrogen from carbon monoxide which makes this process a very expensive method for hydrogen production.

One should know also that natural gas can be transported by pipelines all over Europe and North America. Similar infrastructure could be used to transport hydrogen. Also hydrogen and ethane could be transported in the same pipeline as methane. Hydrogen can be used in fuel cells as a “green” energy carrier with just water as by product. Hydrogen is a clear gas with no color, no odor, non-corrosive and very energetic with 1 kg of hydrogen equivalent with 3 kg of Gasoline and 2.4 kg of methane.

Surprisingly, hydrogen is one of the most abundant elements on the earth but not as molecular H₂. It is present in the sea as water molecule. Nevertheless, at this moment, there is no cheap and or economical way to split water (photo catalytic water spitting and photo-electro catalytic water splitting electrolysis are far from being commercial). Besides water, the most abundant hydrogen containing element on earth is natural gas which contains mainly methane.

One of the more promising alternative technologies to produce hydrogen appears to be the thermal decomposition of methane, also called thermal cracking of methane. In this method, methane can be thermally decomposed to solid carbon and hydrogen. When achieved, this one step process is technologically simple. One of the biggest advantages of methane cracking is the reduction and near elimination of greenhouse gas emissions. However, thermal decomposition of methane typically requires temperatures greater than 1300° C. for complete conversion of methane to solid carbon and hydrogen. An alternative approach consists of the use of a catalyst that can reduce the operating temperatures of the process and increase the rate of methane decomposition which greatly improves the economics of the process and increases the yield of hydrogen. This type of methane cracking is called the thermocatalytic decomposition of methane. The thermocatalytic decomposition of methane was widely reported in the literature since the early 1960s. Despite over fifty years of research, several challenges have also been reported in the literature with the use of the thermocatalytic decomposition of methane. The challenges include greenhouse gas emissions during the regeneration of the catalyst, contamination of the hydrogen produced with carbon oxides, short life time of the catalyst, and production of a wide variation of carbon by-products that cannot always be controlled.

Ethane has also a great potential as a chemical and petrochemical feedstock. One of the most important uses of ethane is in the chemical industry to produce ethylene by steam cracking. As natural gas (methane) is cheap, abundant, and readily available, it would be advantageous to convert methane directly into ethane. The selective non-oxidative coupling of methane into ethane has been disclosed in the literature (see, for example, WO03/104171 and WO2009/115805). Despite all substantial research efforts into non-oxidative methane homologation, such conversion of methane into ethane does not appear to have become a commercial process yet, essentially due to the low efficiency of the current methods.

Thus, there is a need in the art for a hydrogen generation process or a methane conversion process into valuable products that would solve the above identified problems.

### SUMMARY

Methane, which is the main constituent of natural gas, is one of the most widespread sources of hydrogen and carbon in the world. At times, it can be useful to couple methane into ethane in order to use the gas for other purposes. At other times, it can be useful to decompose methane directly into hydrogen and carbon. Advantageously, development of an efficient catalyst that can decompose methane into both hydrogen and solid carbon products, such as carbon black or carbon nanotubes, or methane into ethane, in a selective and controllable manner, can improve economy of hydrogen production.

In one aspect, a method of selectively producing hydrogen or ethane from methane includes selecting a temperature suitable for a metal catalyst and a feed gas including methane to produce a product having a controlled hydrogen/ethane ratio, predominately hydrogen and a solid carbon product or predominately ethane and hydrogen and contacting the feed gas with the metal catalyst at the selected temperature to produce the product.

In another aspect, a method of producing hydrogen includes contacting a feed gas including methane with a ruthenium nanoparticle on a silica nanoparticle support at a temperature suitable to produce a product gas including hydrogen.

In another aspect, a method of selectively producing hydrogen or ethane includes selecting a first pressure and a first temperature suitable to produce hydrogen from methane or a second pressure and a second temperature suitable to produce ethane from methane and contacting a feed gas including methane with a metal catalyst at the selected temperature and selected pressure to produce a product gas including hydrogen or ethane.

In certain embodiments, the selected temperature can be a temperature suitable to produce a product having a hydrogen/ethane ratio of at least 3, at least 5, at least 25, at least 250 or at least 600. In certain other embodiments, the selected temperature can be less than 1000° C., less than 800° C., or greater than 300° C. Selecting the temperature can include choosing a first temperature for the metal catalyst and the feed gas to produce a product gas consisting essentially of hydrogen or a second temperature for the metal catalyst and the feed gas to produce a product gas consisting essentially of ethane and hydrogen.

In certain embodiments, metal catalyst can include ruthenium, nickel, iron, copper, cobalt, palladium, platinum, or combinations thereof. The metal catalyst can be supported on a solid support. The solid support can include a silicon oxide, aluminum oxide, titanium oxide, zirconium oxide, magnesium oxide, cerium oxide, zinc oxide, molybdenum oxide, iron oxide, nickel oxide, cobalt oxide or graphite.

In certain embodiments, the method can include separating the hydrogen from the solid carbon product.

The feed gas can include less than 1000 ppm water or less than 1000 ppm oxygen containing compounds. The feed gas can consist essentially of methane and an inert gas.

The method described herein can increase selectivity and efficiency of methane conversion compared to competitive processes of oxidative coupling, thermal coupling, plasma coupling and non-oxidative catalytic coupling, which are not selective and often require a great deal of energy or temperatures in excess of 1000° C. While thermal decomposition of methane results in production of solid carbon products and hydrogen and can reduce or eliminate greenhouse gas emission, this process typically can require temperatures greater than 1300° C. for complete conversion. A system that allows for decomposition of methane to hydrogen and solid carbon products in a selective manner can significantly improve the commercial viability of methane conversion.

Other aspects, embodiments, and features will be apparent from the following description, the drawings, and the claims.

### BRIEF DESCRIPTION OF THE DRAWINGS

FIG. 1 is a graph depicting thermodynamic minimization of Gibbs free energy assuming a system with the following components; CH₄ (gas), C₂H₆ (gas), H₂ (gas), and C (graphite) at 1 bar, 30 bar and 50 bar.

### DETAILED DESCRIPTION

At the moment, in Europe, hydrogen is largely produced via steam reforming of methane with 60 million tons of hydrogen produced and with 500 million tons of CO₂. This corresponds to 2% of the world emission of CO₂. Moreover, there are already ten hydrogen pipelines in the world mainly in the Netherlands, Belgium and France. This hydrogen can also be transported by boat, supertanker, large cylinders and roads or by pipelines. Ethane is also a good vector for energy and the association of ethane and hydrogen is important, flexibility in the production of hydrogen and ethane is also important regarding transportation of these two gases. Although hydrogen can explode as can propane or gasoline, it has very high diffusivity in the air so that as soon as it is produced it can be diluted easily, which can improve the safety of its use. Indeed, many companies are considering the use of hydrogen either in combustion engine or better as new energy source for fuel cell (e.g. in cars). For example, for an average car trip of 500 km range, the corresponding and respective energy storage expressed in kg is the following: 33 kilos of conventional fuel; 540 kilos of lithium battery, or 6 kg (at 700 bar) of hydrogen.

Methane can be selectively coupled to form ethane or selectively decomposed to form hydrogen and a solid carbon product depending on reaction conditions, such as temperature and pressure. These two processes are commonly known as non-oxidative coupling and thermo-catalytic decomposition of methane, respectively. Surprisingly, a methane coupling catalyst can also be active in thermal decomposition of methane under different sets of operating conditions. Advantageously, ethane present with the hydrogen is easy to separate.

The context of new energy vectors in the next century shows that large scale practical solutions with low carbon dioxide foot print are really a problem. Therefore, the hydrogen generation methodology is extremely timely. Its quick development in the next 20 years will allow emerging technology to become practically feasible. Unexpectedly, methane can be catalytically coupled to ethane and hydrogen at relatively low temperature or to a higher amount of hydrogen than ethane at higher temperature.

Moreover, the formation of hydrogen and ethane does not give carbon dioxide but just carbon, which by its structure can have added value as carbon black, carbon graphite, carbon fiber, or carbon nanotube. Valorization of carbon is extremely important and can be diversified, giving the carbon product having an added value to the process of methane production.

Catalysts and reaction conditions suitable to select between the two reactions can allow for synthetic flexibility, which can lead to clean and efficient generation of hydrogen and/or solid carbon products. Importantly, the catalyst and reaction conditions can be selected to avoid rapid deactivation of the catalyst while maintaining high selectivity for hydrogen production. In certain embodiments, the structure of the solid carbon product can be controlled by selecting the temperature, pressure and catalyst used in the reaction. The solid carbon product can be carbon black, graphene, carbon microfibers, carbon nanofibers, fullerenes, carbon nanotubes (CNTs), single-walled carbon nanotubes, multi-walled carbon nanotubes, or capped carbon nanotubes.

A feed gas including methane is contacted with a metal catalyst at a selected temperature to produce a selected product. In the method, contacting methane with a metal catalyst can include adding the methane to the metal catalyst, adding the metal catalyst to the methane, or by simultaneously mixing the methane and the metal catalyst. In the method, methane can react essentially with itself to couple to form ethane, or form hydrogen and a solid carbon product depending on reaction conditions using a single metal catalyst. Advantageously, the method can produce a product including hydrogen or ethane without forming detectable amounts of carbon-containing products other than alkanes, for example of alkenes (e.g. ethylene), of alkynes (e.g. acetylene), of aromatic compounds (e.g. benzene), of carbon monoxide and/or of carbon dioxide.

The feed gas including methane can contain at least 1%, at least 10%, or at least 20% methane combined with an inert gas, such as nitrogen, helium or argon. The mole ratio of methane to catalyst can be from about 10:1 to 100,000:1, from about 50:1 to 10,000:1, or from about 100:1 to 1,000:1. The feed gas can be dry, having less than 1000 ppm, less than 100 ppm or less than 10 ppm water. The feed gas can include less than 1000 ppm water or other oxygen containing compound, such as an alcohol, carbon monoxide or carbon dioxide.

The method can be carried out at a selected temperature of about 1200° C. or less, about 1000° C., greater than about 300° C., greater than about 400° C., greater than about 500° C., greater than about 600° C., from about 600° C. to about 900° C., from about 650° C. to about 800° C. The temperature is selected to favor production of hydrogen and a solid carbon product from methane or production of ethane from methane. The ratio of hydrogen to ethane produced can vary with temperature.

The method can be carried out at a selected pressure of about 0.1 to about 100 bar, about 0.5 to about 50 bar, about 1 bar, about 5 bar, about 10 bar, about 15 bar, about 20 bar, about 25 bar, about 30 bar, about 35 bar, about 40 bar, or about 45 bar. The pressure is selected to favor production of hydrogen and a solid carbon product from methane or production of ethane from methane. The ratio of hydrogen to ethane produced can vary with pressure.

The method can be carried out as a batch or continuous process. The method can be carried out in a gas phase or a liquid phase system. For example, a fluidized bed reactor and/or a reactor with a mechanically stirred bed can be used. Alternatively, a stationary bed reactor or circulating bed reactor can be used. The gas phase of the product can be continuously removed from the reactor.

The metal catalyst can include at least one metal. In some embodiments, the metal catalyst can include two metals. The metal can be a transition metal, for example, ruthenium, nickel, iron, copper, cobalt, palladium, platinum, or combinations thereof. The catalyst can include a metal combined with a metal oxide, such as its own metal oxide. The metal can be a bimetallic or multi-metallic mixture or alloy. The catalyst can be activated by reduction with hydrogen at a temperature of between 200 and 600° C. for a number of hours. Suitable catalysts are described, for example, in WO2011/107822, which is incorporated by reference in its entirety.

The metal can be on a solid support. The metal can be deposited on a surface of the solid support, covalently bonded to the surface of the solid support, or entrapped within the solid support. The solid support can, for example, be chosen from metal oxides, refractory oxides and molecular sieves, in particular from silicon oxides, aluminum oxides, zeolites, clays, titanium oxide, cerium oxide, magnesium oxide, niobium oxide, zinc oxide, molybdenum oxide, iron oxide, cobalt oxide, tantalum oxide or zirconium oxide. The metal catalyst can include a metal hydride.

The metal of the metal catalyst, or the support, or both, can have nanoscale features. For example, the metal can be in the form of metal nanoparticles having average diameters of less than 200 nm, for example, 5 nm, 10 nm, 15 nm, 20 nm, 30 nm, 40 nm, or 50 nm. The nanoparticles can be spherical or aspherical. The support can have nanoscale features of less than 200 nm, for example, 5 nm, 10 nm, 15 nm, 20 nm, 30 nm, 40 nm, or 50 nm. The nanoparticles can be spherical or aspherical. The support can be, for example, a silica nanoparticle. Suitable nanoparticles can be prepared as described in V. Polshettiwar, et al., Angew. Chem. Int. Ed. 2010, 49, 9652-9656, which is incorporated by reference in its entirety.

Methane decomposition is an endothermic process. Introduction of high temperature condition in the reactor system improves the carbon accumulation and increases the methane conversion by switching the equilibrium to the right. Nevertheless, high temperature condition is subjected to faster deactivation of catalyst. To keep the stability of the catalyst, lower reaction temperature is applied or with diluted methane, but these reduce the catalytic activity.

Reaction temperature can have a great influence on catalyst activity, catalyst lifetime and morphology of the solid carbon product that is produced. Temperature elevation can result in a disproportionately rapid catalyst deactivation. At high temperature, the catalyst can be in a quasi-liquid state where the catalyst particles are easily cut into small particles and the small particles that can be easily encapsulated by the carbon layer formed during methane decomposition, contributing to faster catalyst deactivation. At low temperature, the catalyst remains in solid state rather than in quasi-liquid state and it sustains the activity of catalysis process. Selection of the proper catalyst material can result in catalyst surfaces that do not foul from carbon deposition during the process. In certain examples, ruthenium catalysts are particularly suitable to avoid fouling from carbon deposition.

Carbon nanotube production can be preferable at moderate temperature in order to prolong the catalyst lifetime, but can result in low methane conversion. Low methane conversion can be addressed by separation of the methane-hydrogen mixture at the reactor effluent, followed by recycling of methane. Alternatively, a membrane reactor can be used to remove continuously produced hydrogen from methane decomposition reaction. This alternative can increase methane conversion and enhance the lower temperature reaction. Separation of methane from hydrogen product can increase the operation cost and the hydrogen permeating membrane makes the reactor structure complex. This catalyst system and the optimum operating conditions are expected to contribute effectively towards large-scale production of carbon nanotubes and hydrogen through methane decomposition reaction by using methane gas as carbon source.

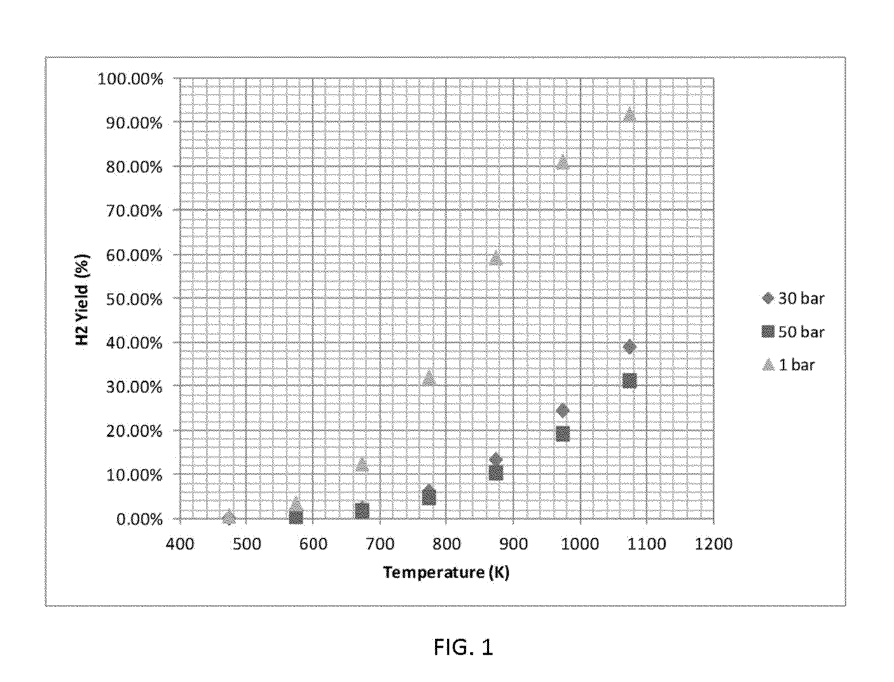

Thermodynamics calculations based on the minimization of Gibbs free energy assuming a system with the following components; CH₄ (gas), C₂H₆ (gas), H₂ (gas), and C (graphite) was carried out at various pressures. The results of the thermodynamics calculation at 1 bar, 30 bar and 50 bar are shown in FIG. 1.

### Examples

### Catalyst Preparation

Preparation of KCC—1-NH₂:

To a 25 mL round-bottom flask, 150 mL of anhydrous toluene, 12.00 g KCC-1, and 40 mL of 3-aminopropyltriethoxysilane (APTS) were successively introduced. The mixture was refluxed for 48 h. The solution was filtered, the solid was washed with acetone and chloroform, and the solid was dried overnight at 65° C. under vacuum to yield the KCC—1-NH₂ nano-composite. Synthesis of suitable catalysts are described, for example, in WO2011/107822, which is incorporated by reference in its entirety.

Preparation of Catalysts (KCC—1-NH₂/Ru NPs, KCC—1-NH₂/Fe NPs, KCC—1-NH₂/Co NPs):

A Schlenk flask was charged with 3 g of KCC—1-NH₂ material and the required amount of metal chloride (e.g. RuCl₃, FeCl₂, CoCl₂), (e.g., 0.21 g of RuCl₃) was sonicated in 50 ml of deionized water for 2 h. The mixture was stirred for 72 h at room temperature. The solid was collected by centrifugation and washed several times with water, ethanol and acetone. The solid was then dried under reduced pressure at 65° C. for 16 h, which resulted in a grey powder (3.2 g). The reduction was performed in a fixed-bed continuous flow reactor. For the in situ preparation of the ruthenium nanoparticles, the unreduced catalyst (200 mg) was placed in a stainless steel tubular reactor with a 9-mm internal diameter and was reduced in a stream of hydrogen (20 mL/min) at 400° C. for 16 h. The ruthenium content of the final material was determined by ICP elemental analysis and was found to be 4.2%.

### Catalytic Tests

The catalytic tests for methane coupling and/or decomposition were carried out in a fixed-bed continuous flow reactor. The powdered catalyst was charged in a stainless steel tubular reactor that was placed in an electric furnace. The temperature in the reactor was controlled by a PID temperature controller connected to the thermocouple placed inside catalyst bed and maintained with a frit.

The catalytic activity was determined by filing the reactor with N₂ until reaching 30 bar. Methane was allowed to pass over the catalyst at a rate varied between 3 and 12 mL/min. The individual gas flow rates were controlled using mass flow controllers, previously calibrated for each specific gas. The activity of the catalyst was tested continuously several hours, by keeping the catalyst at a constant temperature, until the conversion is stabilized.

The feed gases and the products were analyzed employing an online Gas Chromatograph equipped with TCD and FID detectors using He and H₂ as a carrier gases respectively.

First of all, the idea was to test catalysts in the reaction of methane coupling (equation 1) and to study the effect of the temperature under isobar conditions (30 bars).

By definition, the conversion of methane is

$$conversion = \frac{n ( CH 4 ) in - n ( CH 4 ) out}{n ( CH 4 ) in}$$

Assuming the following two reactions

2CH₄→C₂H₆+H₂

CH₄→C₍ₛ₎+2H₂

From carbon balance, The number of moles of methane introduced should be equal to

n(CH4)in=n(CH4)+2n(C2H6)+n(c)

However, n(c) is unknown, but can be estimated as follows;

The total number of moles of H₂ in the gas phase is

n(H2)=n(H2)coupling+n(H2)MD; MD=methane decomposition

$$n ( H 2 ) coupling = n ( C 2 H 6 )$$

$$n ( H 2 ) MD = 2 n ( C )$$

$$n ( c ) = \frac{n ( H 2 ) - n ( C 2 H 6 )}{2}$$

$$conversion = \frac{1.5 n ( C 2 H 6 ) + 0.5 n ( H 2 )}{n ( CH 4 ) + 1.5 n ( C 2 H 6 ) + 0.5 n ( H 2 )}$$

Assuming that there is no significant change in total number of moles before and after reaction;

$$conversion = \frac{1.5 x ( C 2 H 6 ) + 0.5 x ( H 2 )}{x ( CH 4 ) + 1.5 x ( C 2 H 6 ) + 0.5 x ( H 2 )} ;$$

x=molar fraction determined from GC

Yield of H₂:

Each mole of CH₄ gives ideally a maximum of 2 moles of H₂.

Thus,

$$H 2 yield ( \% ) = \frac{n ( H 2 )}{2 n ( CH 4 ) in}$$

$$H 2 yield ( \% ) = \frac{n ( H 2 )}{2 \times ( n ( CH 4 ) + 1.5 n ( C 2 H 6 ) + 0.5 n ( H 2 ) )}$$

Yield of C2:

Each mole of CH₄ gives ideally a maximum of 0.5 moles of C₂H₆,

Thus,

$$C 2 yield ( \% ) = \frac{n ( C 2 H 6 )}{0.5 n ( CH 4 ) in}$$

$$C 2 yield ( \% ) = \frac{n ( C 2 H 6 )}{0.5 \times ( n ( CH 4 ) + 1.5 n ( C 2 H 6 ) + 0.5 n ( H 2 ) )}$$

### Catalyst Runs

Reactions were carried out using as a catalyst KCC-1/Ru nanoparticles, 4.1 wt % Ru. Unless otherwise noted, the reactions used 200 mg catalyst, pressure 29 bar, methane flow of 3 ml/min.

The data in Table 1 were for reactions carried out regeneration of catalyst (15 h at 400° C. under an H₂ flow of 20 ml/min).

TABLE 1

| Conditions | | | Experimental | | | | Thermodynamics | | | |
|---|---|---|---|---|---|---|---|---|---|---|
| Catalyst | | | Molar | | C₂ | H₂ | | | | C2 |
| loading | Temperature | CH₄ | Ratio | Conversion | Yield | Yield | Conversion | | H2 | Yield |
| (mg) | (° C.) | (ml/min) | (H₂/C2) | (%) | (%) | (%) | (%) | H2/C2 | yield | (%) |
| 200 | 400 | 3 | 5.65 | 0.32 | 0.16 | 0.2 | 2.3 | 316 | 2.3 | 0.01 |
| 200 | 400 | 3 | 5.33 | 0.31 | 0.14 | 0.2 | 2.3 | 316 | 2.3 | 0.01 |
| 200 | 500 | 3 | 45.59 | 1.14 | 0.1 | 1.07 | 6.1 | 557 | 6.1 | 0.02 |
| 200 | 400 | 3 | 5.58 | 0.28 | 0.13 | 0.18 | 2.3 | 316 | 2.3 | 0.01 |
| 200 | 400 | 6 | 3.66 | 0.22 | 0.13 | 0.12 | 2.3 | 316 | 2.3 | 0.01 |
| 200 | 400 | 9 | 5.36 | 0.22 | 0.11 | 0.13 | 2.3 | 316 | 2.3 | 0.01 |
| 200 | 400 | 12 | 7.31 | 0.08 | 0.01 | 0.07 | 2.3 | 316 | 2.3 | 0.01 |
| 500 | 400 | 6 | 3.72 | 0.24 | 0.14 | 0.13 | 2.3 | 316 | 2.3 | 0.01 |
| 500 | 400 | 6 | 3.13 | — | — | — | 2.3 | 316 | 2.3 | 0.01 |
| 500 | 600 | 3 | 290.09 | 5.08 | 0.07 | 5.03 | 13.3 | 1987 | 13.3 | 0.03 |
| 500 | 700 | 3 | 857.05 | 12.7 | 0.06 | 12.6 | 24.5 | 3090 | 24.5 | 0.03 |

It is notable that the experimental yields of C2 are higher than those expected from thermodynamics.

Additional experiments using iron or cobalt metals as catalysts were conducted, and the results are summarized in Table 2.

TABLE 2

| | Temp | Time | CH₄ | H₂/C₂H₆ | Conversion | C₂H₆ yield | H₂ yield | C yield |
|---|---|---|---|---|---|---|---|---|
| catalyst | (° C) | (h) | (ml/min) | molar ratio | (%) | (%) | (%) | (%) |
| KCC-1/Fe | 600 | 24 | 3 | n/a | 3.23 | Not | 2.16 | 1.08 |
| NPs | | | | | | measured | | |
| | 700 | 24 | 3 | n/a | 23.70 | Not | 15.80 | 7.90 |
| | | | | | | measured | | |
| | 800 | 24 | 3 | n/a | 99.34 | Not | 66.23 | 33.11 |
| | | | | | | measured | | |
| KCC-1/Co | 600 | 24 | 3 | n/a | 0.68 | Not | 0.45 | 0.23 |
| NPs | | | | | | measured | | |
| | 700 | 24 | 3 | n/a | 8.48 | Not | 5.65 | 2.83 |
| | | | | | | measured | | |
| | 800 | 24 | 3 | n/a | 99.77 | Not | 66.52 | 33.26 |
| | | | | | | measured | | |

The obtained results indicated two parallel competitive reactions can take place: i) coupling of methane into ethane, and ii) thermal decomposition of methane to hydrogen and carbon.

Other embodiments are within the scope of the following claims.

What is claimed is:

1. A method of selectively producing hydrogen or ethane from methane comprising:
selecting a temperature suitable for a metal catalyst and a feed gas including methane to produce a product having a controlled hydrogen/ethane ratio, predominately hydrogen and a solid carbon product or predominately ethane and hydrogen;
contacting the feed gas with the metal catalyst at the selected temperature to produce the product.

2. The method of claim 1, wherein the selected temperature is a temperature suitable to produce a product having a hydrogen/ethane ratio of at least 3.

3. The method of claim 1, wherein the selected temperature is a temperature suitable to produce a product having a hydrogen/ethane ratio of at least 5.

4. The method of claim 1, wherein the selected temperature is a temperature suitable to produce a product gas having a hydrogen/ethane ratio of at least 25.

5. The method of claim 1, wherein the selected temperature is a temperature suitable to produce a product gas having a hydrogen/ethane ratio of at least 250.

6. The method of claim 1, wherein the selected temperature is a temperature suitable to produce a product gas having a hydrogen/ethane ratio of at least 600.

7. The method of claim 1, wherein the selected temperature is less than 1000° C.

8. The method of claim 1, wherein the selected temperature is less than 800° C.

9. The method of claim 1, wherein the selected temperature is greater than 300° C.

10. The method of claim 1, wherein the metal catalyst includes ruthenium, nickel, iron, copper, cobalt, palladium, platinum, or combinations thereof.

11. The method of claim 1, wherein the metal catalyst is supported on a solid support.

12. The method of claim 11, wherein the solid support includes a silicon oxide, aluminum oxide, titanium oxide, zirconium oxide, magnesium oxide, cerium oxide, zinc oxide, molybdenum oxide, iron oxide, nickel oxide, cobalt oxide or graphite.

13. The method of claim 1, further comprising separating the hydrogen from the solid carbon product.

14. The method of claim 1, wherein the feed gas comprises less than 1000 ppm water.

15. The method of claim 1, wherein the feed gas consists essentially of methane and an inert gas.

16. The method of claim 1, wherein the feed gas includes less than 1000 ppm oxygen containing compounds.

17. The method of claim 1, wherein selecting the temperature includes choosing a first temperature for the metal catalyst and the feed gas to produce a product gas consisting essentially of hydrogen or a second temperature for the metal catalyst and the feed gas to produce a product gas consisting essentially of ethane and hydrogen.

18. A method of producing hydrogen comprising contacting a feed gas including methane with a ruthenium nanoparticle on a silica nanoparticle support at a temperature suitable to produce a product gas including hydrogen.

19. A method of selectively producing hydrogen or ethane comprising selecting a first pressure and a first temperature suitable to produce hydrogen from methane or a second pressure and a second temperature suitable to produce ethane from methane and contacting a feed gas including methane with a metal catalyst at the selected temperature and selected pressure to produce a product gas including hydrogen or ethane.
